SEQUENCE LISTING (6 sequences)

SEQ ID NO: 1 - DNA, 53 bp, synthetic construct
  source 1..53: mol_type = other DNA
tcaacatcactggagatctaactcatggtacctgttagatctccagttttttt

SEQ ID NO: 2 - DNA, 54 bp, synthetic construct
  misc_feature 1..37: note = DNA
  misc_feature 37..54: note = RNA
  source 1..54: mol_type = other DNA
gcagttgatcctttggataccctggggattggttttgggtatccaaaggatcaa

SEQ ID NO: 3 - DNA, 35 bp, synthetic construct
  misc_feature 11..13: note = RNA
  source 1..35: mol_type = other DNA
  misc_feature <11..>13: note = DNA
tcaacatcactttgttagatctccagtgatgttga

SEQ ID NO: 4 - RNA, 48 nt, synthetic construct
  source 1..48: mol_type = other RNA
gaccaccccaaaaatgaaggggactaaaacttgatcctttggataccc

SEQ ID NO: 5 - DNA, 52 bp, synthetic construct
  source 1..52: mol_type = other DNA
gatctaacaggtaccatgagttagatctccagttcatggtacctcgactcat

SEQ ID NO: 6 - DNA, 16 bp, synthetic construct
  source 1..16: mol_type = other DNA
ttttttatgagtcgag

(19) United States
(12) Patent Application Publication
(10) Pub. No.: US 2026/0146955 A1
(43) Pub. Date: May 28, 2026

(54) SERS BIOSENSOR BASED ON CRISPR/CAS13A SYSTEM AND APPLICATION THEREOF

(71) Applicant: NANJING UNIVERSITY OF POSTS AND TELECOMMUNICATIONS, NANJING (CN)

(72) Inventors: CHUNYUAN SONG, NANJING (CN); LIANHUI WANG, NANJING (CN)

(73) Assignee: NANJING UNIVERSITY OF POSTS AND TELECOMMUNICATIONS, NANJING (CN)

(21) Appl. No.: 19/342,568

(22) Filed: Sep. 28, 2025

Related U.S. Application Data
(63) Continuation of application No. PCT/CN2024/135334, filed on Nov. 28, 2024.

(51) Int. Cl. G01N 21/65; C12N 9/22; C12N 15/113; G01N 33/50

(52) U.S. Cl. CPC G01N 21/658; C12N 9/226; C12N 15/113; G01N 33/50; C12N 2310/122; C12N 2310/16; C12N 2310/20; C12N 2320/10; G01N 2333/922; G01N 2800/7028

(57) ABSTRACT

Provided are a Surface-Enhanced Raman Scattering (SERS) biosensor based on a Clustered Regularly Interspaced Short Palindromic Repeats (CRISPR)/Cas13a system and an application thereof, belonging to the field of spectroscopy detection. The SERS biosensor includes an SERS sensor chip, a first reagent, a second reagent, a third reagent, and a fourth reagent. The SERS sensor chip is a silver nanorod array substrate with hairpin Deoxyribonucleic Acid (DNA) single-stranded H1 and blocking molecule 6-mercaptoethanol modified on the surface. The first reagent is a SERS probe and buffer of the probe. The second reagent is hairpin nucleic acid aptamer strand MUC1-apt aqueous solution. The third reagent is the CRISPR/Cas13a system and buffer of the system. The fourth reagent is hairpin DNA single-stranded H2 aqueous solution. The above reagents are mixed with the gastric cancer exosomes to be detected and then dripped on a surface of the SERS sensor chip.

### CROSS-REFERENCE TO RELATED PRESENT DISCLOSURE

This application is a continuation of International Application No. PCT/CN2024/135334, filed on Nov. 28, 2024, which claims priority to Chinese Patent Application No. 202410533048.2, filed on Apr. 30, 2024. The disclosures of the above-mentioned applications are hereby incorporated by reference in their entireties.

### INCORPORATION BY REFERENCE OF SEQUENCE LISTING

The content of the following submission is incorporated herein by reference in its entirety: a computer-readable format (CRF) of the Sequence Listing (file name: Sequence_Listing.xml, date recorded: Apr. 30, 2024, size: 7,784 bytes).

### TECHNICAL FIELD

The present disclosure belongs to the field of spectroscopy detection, and in particular, relates to a Surface-Enhanced Raman Scattering (SERS) biosensor based on a Clustered Regularly Interspaced Short Palindromic Repeats (CRISPR)/Cas13a system and an application thereof.

### BACKGROUND

Gastric cancer is one of the most common malignant tumors in the world. In 2020, the incidence of gastric cancer in the world ranks fifth, and the mortality rate ranks fourth. Most patients have no obvious symptoms in the early stage of gastric cancer, and the disease is often diagnosed at an advanced stage once symptoms appear. The clinical diagnosis of gastric cancer mainly depends on endoscopic biopsy and medical imaging technology, and the detection rate of early tumor micro-lesions is low. Therefore, early diagnosis of gastric cancer is an urgent task for formulating more effective treatment strategies and reducing the mortality rate.

An exosome is an extracellular vesicle, which is involved in physiological processes, such as intercellular substance transfer, information exchange, cell proliferation and differentiation, and is also related to many pathological processes, such as tumor invasion and metastasis. Therefore, exosomes from cancer have great potential to become biomarkers for early clinical diagnosis and evaluation of a cancer treatment effect. Conventional exosome detection methods, such as protein blot, flow cytometry, Nanoparticle Tracking Analysis (NTA) and Enzyme-Linked Immunosorbent Assay (ELISA), and the like. However, the abundance of exosomes from cancer in complex body fluids is extremely low. The conventional detection methods have some problems, such as expensive instruments, complicated operation, time-consuming detection, insufficient sensitivity and accuracy. Therefore, it is urgent to develop a sensitive and reliable exosome detection method to achieve accurate diagnosis of early gastric cancer.

The Surface-Enhanced Raman Scattering (SERS) has been regarded as a powerful analytical tool because of the ultra-high sensitivity and fingerprint specificity. Compared with immunoassay based on antibody recognition, the aptamer shows high binding affinity and specificity to the target, and is easy to synthesize and store, good in stability, and low in immunogenicity and toxicity. In addition, a single-stranded DNA aptamer can be combined with a variety of DNA-based signal amplification reactions, which is expected to significantly improve the sensitivity and specificity of exosome detection.

The US2024101307311, which was previously applied by our research group, provides an SERS biosensor, and a preparation method and an application thereof, which provides a solution for detecting exosomes of early gastric cancer, and improves the detection sensitivity to some extent. However, the overall detection efficiency is not high because samples need to be pre-processed in the detection process.

### SUMMARY

Objective of the present disclosure: In view of the great demand for rapid, sensitive and accurate determination in exocrine detection at present, and the shortcomings of conventional detection methods such as low sensitivity, poor specificity, and time-consuming detection, the present disclosure discloses a Surface-Enhanced Raman Scattering (SERS) biosensor based on a Clustered Regularly Interspaced Short Palindromic Repeats (CRISPR)/Cas13a system and an application thereof in combination with the CRISPR/Cas13a system and the cascade signal amplification strategy of Catalytic Hairpin Assembly (CHA) based on the aptamer recognition technology. The SERS biosensor is simple in preparation, requires no amplification for detection, and does not need to be operated by professional technicians. The SERS biosensor is rapid in detection (60 minutes), high in sensitivity (the limit of detection is as low as the level of 10² particles·mL⁻¹), and good in specificity (exosomes from other cells can be clearly distinguished), which achieves the rapid, sensitive and accurate determination of gastric cancer exosomes and provides an innovative and feasible solution for the diagnosis of early gastric cancer.

In order to solve the technical problems, the present disclosure uses the following technical solution.

In a first aspect, the present disclosure provides a Surface-Enhanced Raman Scattering (SERS) biosensor based on a Clustered Regularly Interspaced Short Palindromic Repeats (CRISPR)/Cas13a system, including an SERS sensor chip, a first reagent, a second reagent, a third reagent, and a fourth reagent.

As shown in FIG. 1A, the SERS sensor chip is a silver nanorod array substrate with hairpin Deoxyribonucleic Acid (DNA) single-stranded H1 and a blocking molecule 6-mercaptoethanol (MCH) modified on the surface. The hairpin DNA single-stranded H1 is connected to the surface of the substrate by forming an Ag—S covalent bond with the silver nanorod, and 6-mercaptoethanol (MCH) aqueous solution is dripped onto the surface of the substrate for blocking, which can reduce nonspecific adsorption.

The first reagent is an SERS probe and buffer of the probe. As shown in FIG. 1B, the SERS Probe is a composite nanoparticle where a surface of a gold nanoparticle is modified with a Probe single strand and a Raman molecule 5,5′-dithiobis(2-nitrobenzoic acid) (DTNB). Preferably, the buffer of the SERS probe is 0.5×TBE.

The second reagent is hairpin nucleic acid aptamer strand MUC1-apt aqueous solution. Preferably, the configuration concentration of the hairpin nucleic acid aptamer strand MUC1-apt aqueous solution is 5 to 20 μM.

The third reagent is the CRISPR/Cas13a system and buffer of the system, and the CRISPR/Cas13a system includes Cas13a protein, crRNA, and uracil-modified hairpin recognition single-stranded HR. Preferably, the configuration concentrations of the Cas13a protein and the crRNA are both 0.1 to 2 μM, and the configuration concentration of the uracil-modified hairpin recognition single-stranded HR is 1 to 10 μM.

The fourth reagent is hairpin DNA single-stranded H2 aqueous solution. Preferably, the configuration concentration of the hairpin DNA single-stranded H2 is 5 to 20 μM.

The hairpin DNA single-stranded H1 base sequence is shown in SEQ ID NO. 1, the hairpin nucleic acid aptamer strand MUC1-apt base sequence is shown in SEQ ID NO. 2, the uracil-modified hairpin recognition single-stranded HR base sequence is shown in SEQ ID NO. 3, the crRNA base sequence is shown in SEQ ID NO. 4, the hairpin DNA single-stranded H2 base sequence is shown in SEQ ID NO. 5, and the Probe single-stranded base sequence is shown in SEQ ID NO. 6.

In some embodiment, the particle size of the gold nanoparticle is 15 to 100 nm. Further preferably, the particle size of the gold nanoparticle is 15 nm.

Preferably, the preparation method of the SERS sensor chip is as follows:
(1) preparing a silver nanorod array, and washing the silver nanorod array with Diethyl Pyrocarbonate (DEPC) water for many times before use;
(2) co-culturing the hairpin DNA single-stranded H1 aqueous solution with the silver nanorod array in the concentration range of 0.5 to 2 μM, and standing for 3 to 5 hours at the culturing condition of 25 to 37° C. and the humidity environment of 60 to 80%; where H1 is immobilized on the surface of the silver nanorod array through a covalent bond formed between a thiol group and a silver; and further preferably, the concentration of the hairpin DNA single-stranded H1 aqueous solution is 0.5 μM; and
(3) after cleaning the substrate with reaction buffer with pH of 8.3, dripping 10 μM of 6-mercaptoethanol (MCH) aqueous solution onto a surface of the substrate, and placing MCH aqueous solution in a constant temperature mixer at 37° C. for 10 minutes; where a volume ratio of the hairpin DNA single-stranded H1 aqueous solution to the 6-mercaptoethanol (MCH) aqueous solution is 1:1; and the components of the reaction buffer include 10 mM of Tris-HCl, 50 mM of KCl, and 1.5 M of MgCl₂.

Preferably, the silver nanorod array is prepared by a vacuum electron beam evaporation coating device and an oblique angle deposition technology, and the silver nanorod array substrate is covered with a layer of polydimethylsiloxane (PDMS) film with 3×10 pores, in which a single pore has a diameter of 4 mm and a height of 1 mm.

Preferably, the preparation step of the first reagent includes:
1) mixing the Probe single strand with a Tricarboxyethyl Phosphine Solution (TCEP) according to a molar ratio of 1:1000, and placing the mixture in a constant temperature mixer at 25° C. for reaction for 4 hours;
2) mixing 50 μM of Probe single-stranded aqueous solution with 2.3 nM of AuNPs aqueous solution in Phosphate-Buffered Saline (PBS) buffer with pH 7.4, in which the components of the buffer include 10 mM of Na₂HPO₄, 1.76 mM of KH₂PO₄, 137 mM of NaCl, and 2.7 mM of KCl, and the mixture is placed in the constant temperature mixer at 25° C. for shaking incubation for 5 hours; and a volume ratio of the Probe single-stranded aqueous solution to the AuNPs aqueous solution is 1:50;
3) adding 10, 20, 30 and 40 μL of 2 M of NaCl solution into the mixture obtained in Step 2) in sequence every 0.5 hour for shaking incubation for 5 hours; and
4) adding 100 μM of DTNB to the mixture obtained in Step 3), placing the mixture in the constant temperature mixer at 25° C. for shaking incubation for 3 hours, centrifuging at 12000 rpm for 20 minutes, and washing the mixture with the PBS buffer for three times; and finally, re-dispersing the SERS probe in the PBS buffer and storing the SERS probe at 4° C. for subsequent use.

In a second aspect, the present disclosure provides an application of the Surface-Enhanced Raman Scattering (SERS) biosensor in preparing a detection kit for gastric cancer cell exosomes, where the application step includes:
1) adding SGC-7901 cell-derived exosomes into Phosphate-Buffered Saline (PBS) buffer to prepare sample solution; where the concentration of exosomes in the obtained sample solution ranges from 1.44×10³ to 1.44×10⁷ particles·mL⁻¹;
2) mixing hairpin nucleic acid aptamer strand MUC1-apt, Cas13a protein, crRNA, uracil-modified hairpin recognition single-stranded HR, hairpin DNA single-stranded H2, and an SERS probe with different concentrations of gastric cancer exosome samples, and dripping the mixture on the surface of an SERS sensor chip for co-culturing; and standing for 60 minutes at the culturing condition of 25 to 37° C. and the humidity environment of 60 to 80%; and
3) washing the sample processed in Step 2) with Diethyl Pyrocarbonate (DEPC) water for many times, and then carrying out the SERS test to obtain SERS spectra of target exosomes with different concentrations, drawing working curves of SERS sensors with the logarithm of the target exosome concentration as the abscissa and the SERS intensity of the DTNB characteristic peak as the ordinate, respectively, and calculating the limit of detection of the SERS sensor of the sensor according to the working curves.

Culturing is performed in a constant temperature mixer of 300 rpm in Step 2) for 60 minutes preferably at the culturing condition of 25 to 37° C. and the humidity environment of 60 to 80%.

The detection principle of the present disclosure is as follows.

As shown in FIG. 1C, the SERS biosensor provided by the present disclosure is used to detect gastric cancer exosomes. When there are target exosomes, the recognition sequence in the hairpin nucleic acid aptamer strand MUC1-apt specifically binds, and the exosome membrane protein is recognized, so that the hairpin structure is opened. Another part of the free RNA fragment of MUC1-apt is guided by the crRNA to hybridize with the spacer of the Cas13a/crRNA complex. The conformational change of the RNA protein complex is triggered. Two HEPN structure domains are close to form a catalytic site, which successfully activates the RNA digestion activity of Cas13a protein. The activated CRISPR/Cas13a system can cut any single-stranded RNA, including uracil-modified hairpin recognition single-stranded HR, which can be cut to release ST fragments. Subsequently, the ST fragments can trigger the hairpin DNA single-stranded H1 on the surface of the SERS sensor chip to be opened and form the H1/ST complex. With the assistance of the hairpin DNA single-stranded H2, the Catalytic Hairpin Assembly (CHA) reaction occurs, forming a large number of H1-H2 double strands on the surface of the SERS sensor chip and releasing ST into the next cycle. At the same time, there are still free fragments at the end of the double strands formed by binding H1 and H2, which can be complementary to the Probe strand modified on the SERS probe. The sticky end of the H1-H2 double strands captures the SERS probe to the surface of the SERS sensor chip, so as to output significantly enhanced Raman signals from Raman molecules on the SERS probe and achieve the detection of target exosomes. An exosome in the sample can activate the CRISPR/Cas13a system through MUC1-apt to produce a large number of ST fragments, and the generated ST fragments enter the Catalytic Hairpin Assembly (CHA), thus achieving high-sensitivity detection.

### Beneficial Effects

Compared with the SERS biosensor previously applied by our research group, the SERS biosensor provided by the present disclosure is more convenient for detecting gastric cancer exosomes without pre-processing sample, and can achieve one-step incubation detection of exosomes, improve the detection efficiency, and achieve sensitive detection at the level of 10² particles·mL⁻¹ within 60 minutes.

The SERS biosensor provided by the present disclosure is compared with the immunoassay technology based on antibody recognition, such as documents from Li J., Li Y., Chen S., et al. Highly sensitive exosome detection for early diagnosis of pancreatic cancer using immunoassay based on hierarchical surface-enhanced Raman scattering substrate. Small methods, 2022, 6 (6), 2200154. The SERS biosensor provided by the present disclosure is simple in preparation and application. The sensitive detection at the level of 10² particles·mL⁻¹ can be achieved in a short time (60 minutes), The SERS biosensor has good specificity, uniformity, and repeatability. The present disclosure is suitable for screening high-risk groups of gastric cancer, and can achieve sensitive and accurate clinical diagnosis of patients with early gastric cancer.

### BRIEF DESCRIPTION OF THE DRAWINGS

FIG. 1A is a schematic diagram of the construction of an SERS sensor chip according to the present disclosure;

FIG. 1B is a schematic diagram of the construction of an SERS probe of the present disclosure;

FIG. 1C is a working principle diagram of an SERS biosensor used for detecting gastric cancer exosomes;

FIG. 2A is an SERS spectrogram of a mechanism of an SERS biosensor for detecting gastric cancer exosomes in Embodiment 1;

FIG. 2B is an SERS peak intensity of each spectral line corresponding to the Raman shift of 1331 cm⁻¹ in FIG. 2A;

FIG. 3 is an optimization experiment of the incubation concentration of the hairpin DNA single-stranded H1 on the surface of the SERS sensor chip in a specific embodiment;

FIG. 4 is an optimization experiment of 6-mercaptoethanol (MCH) blocking time on the surface of the SERS sensor chip in a specific embodiment;

FIG. 5 is an optimization experiment of detection time of detecting gastric cancer exosomes by using an SERS biosensor in Experimental Example 1;

FIG. 6A is an SERS spectrogram corresponding to the detection of gastric cancer exosomes with different concentrations by using an SERS biosensor in Experimental Example 2;

FIG. 6B is an SERS peak intensity of each spectral line corresponding to the Raman shift of 1331 cm⁻¹ in FIG. 6A;

FIG. 7A is an SERS spectrogram of an SERS biosensor used to detect mixed samples of GES-1, HepG2, and GES-1/HepG2/SGC-7901 in Experimental Example 3;

FIG. 7B is an SERS peak intensity of each spectral line corresponding to the Raman shift of 1331 cm⁻¹ in FIG. 7A;

FIG. 8 shows results of uniformity characterization of detecting gastric cancer exosomes by using an SERS biosensor in Experimental Example 3; and

FIG. 9 shows results of repeatability characterization of detecting gastric cancer exosomes by using an SERS biosensor in Experimental Example 3.

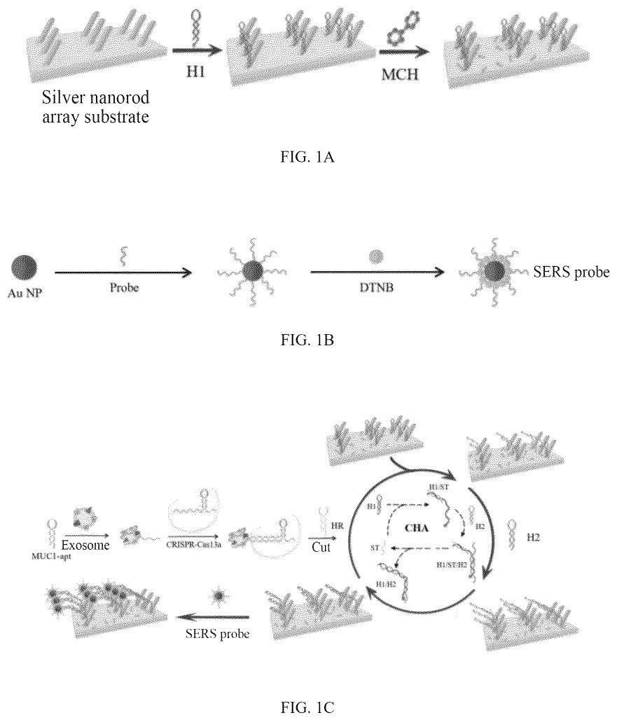

### DETAILED DESCRIPTION OF THE EMBODIMENTS

In order to allow those skilled in the art to better understand the content of the patent of the present disclosure, the embodiment of the present disclosure may be described in detail below. This embodiment is implemented on the premise of the technical solution of the present disclosure, and the detailed implementation and the specific operation process are given, but the content of the present disclosure is not limited to the embodiment.

The nucleic acid base sequence fragments used in the present disclosure are all obtained by artificial synthesis and are all synthesized by Sangon Biotech (Shanghai) Co., Ltd.

The specific configuration of reagents used in the following embodiments is as follows.

The acquisition and configuration process of gastric cancer exosome samples are as follows: the gastric cancer exosome samples are separated from SGC-7901 cells by gradient ultracentrifugation; the intact cells in the supernatant are removed by freezing centrifugation (4° C., 500 g, 10 min), then the cell fragments and apoptotic bodies in the supernatant are removed by centrifugation (4° C., 10000 g, 90 min), and finally, the supernatant is removed by centrifugation (4° C., 10000 g, 120 min), and the centrifugal product is dispersed in the PBS buffer to obtain sample solution. The concentration of exosomes in the obtained sample solution ranges from 1.44×10³ to 1.44×10⁷ particles·mL⁻¹.

As for the source of the second reagent which is hairpin aptamer strand MUC1-apt aqueous solution: MUC1-apt is synthesized by Sangon Biotech (Shanghai) Co., Ltd., and the base sequence of MUC1-apt is 5′-GCA GTT GAT CCT TTG GATACC CTG GGG ATT GGT TTT/rG//rG//rG//rU//rA//rU//rC//rC//rA//rA//rA//rG//rG//rA//rU//rC//rA//rA/-3′.

As for the source of the third reagent which is the CRISPR/Cas13a system: the CRISPR/Cas13a system includes Cas13a protein, crRNA, uracil-modified hairpin recognition single-stranded HR and buffer thereof; where Cas13a protein is available from Guangzhou Bio-Lifescico., Ltd.; CrRNA and HR are synthesized by Sangon Biotech (Shanghai) Co., Ltd.; the base sequence of crRNA is 5′-GAC CAC CCC AAAAAU GAA GGG GAC UAAAAC UUG AUC CUU UGG AUA CCC-3′; the base sequence of HR is 5′-TCAACA TCA C/rU//rU//rU/GTT AGA TCT CCA GTG ATG TTGA-3′, which is the DNA sequence of intermediate modified uracil/rU/; where GTT AGA TCT CCA GTGATG TTGA block is the ST sequence.

As for the source of the fourth reagent which is hairpin DNA single-stranded H2 aqueous solution: H2 is synthesized by Sangon Biotech (Shanghai) Co., Ltd.; and the base sequence of H2 is 5′-GAT CTAACA GGT ACC ATGAGT TAGATC TCCAGT TCATGGTAC CTC GAC TCAT-3′.

As for the preparation of the first reagent which is the SERS probe:
(1) the Probe single-stranded solution is mixed with a Tricarboxyethyl Phosphine Solution (TCEP) according to a molar ratio of 1:1000, and the mixture is placed in a constant temperature mixer at 25° C. for reaction for 4 hours; and the Probe single strand is synthesized by Sangon Biotech (Shanghai) Co., Ltd., and specifically consists of nucleotides and substituents with the following base sequences: 5′-SH—(CH₂)₆-TTT TTTATGAGT CGAG-3′;
(2) 10 μL of 50 μM of TCEP-processed Probe single-stranded aqueous solution is mixed with 500 μL of 2.3 nM of AuNPs aqueous solution in Phosphate-Buffered Saline (PBS) buffer with pH 7.4, and the mixture is placed in the constant temperature mixer at 25° C. for shaking incubation for 5 hours; and the components of the buffer include 10 mM of Na₂HPO₄, 1.76 mM of KH₂PO₄, 137 mM of NaCl, and 2.7 mM of KCl;
(3) 10, 20, 30 and 40 μL of 2 M of NaCl solution is added into the mixture obtained in Step 2) in sequence every 0.5 hour for shaking incubation for 5 hours; and
(4) 50 μL of 100 μM of DTNB is added to the mixture obtained in Step 3), placing the mixture in the constant temperature mixer at 25° C. for shaking incubation for 3 hours, centrifuging is performed (at 12000 rpm for 20 minutes), and the mixture is washed with the PBS buffer for three times. Finally, the SERS probe is re-dispersed in 100 μL of PBS buffer 0.5×TBE and is stored at 4° C. for subsequent use.

As for preparation of the SERS sensor chip:
(1) a silver nanorod array is prepared, see documents from Song C., Chen J., Zhao Y., et al. Gold-modified silver nanorod arrays for SERS-based immunoassays with improved sensitivity. Journal of Materials Chemistry B, 2014, 2 (43): 7488-7494; the silver nanorod array substrate is prepared by the vacuum electron beam evaporation coating technology; the silver nanorod array substrate is covered with a layer of polydimethylsiloxane (PDMS) film with 3×10 pores, in which a single pore has a diameter of 4 mm and a height of 1 mm; and the silver nanorod array is washed with Diethyl Pyrocarbonate (DEPC) water for many times before use;
(2) 20 μL of 500 nM of the hairpin DNA single-stranded H1 aqueous solution is co-cultured with the silver nanorod array, where H1 is immobilized on the surface of the silver nanorod array through a covalent bond formed between a thiol group and a silver; stand for 3 hours at the culturing condition of 37° C. and the humidity environment of 60%; H1 is synthesized by Sangon Biotech (Shanghai) Co., Ltd., and specifically consists of nucleotides and substituents with the following base sequences: 5′-TCAACA TCA CTG GAG ATC TAA CTC ATG GTA CCT GTT AGA TCT CCA GTT TTT TT-SH—(CH₂)₆-3′; and
(3) after cleaning the substrate with reaction buffer (10 mM of Tris-HCl, 50 mM of KCl, 1.5 M of MgCl₂, pH 8.3), 200 μL of 10 μM of 6-mercaptoethanol (MCH) aqueous solution is dripped onto a surface of the substrate, and MCH aqueous solution is placed in a constant temperature mixer at 37° C. for 10 minutes.

As for optimization of incubation concentration of hairpin DNA single-stranded H1 on the surface of the SERS sensor chip

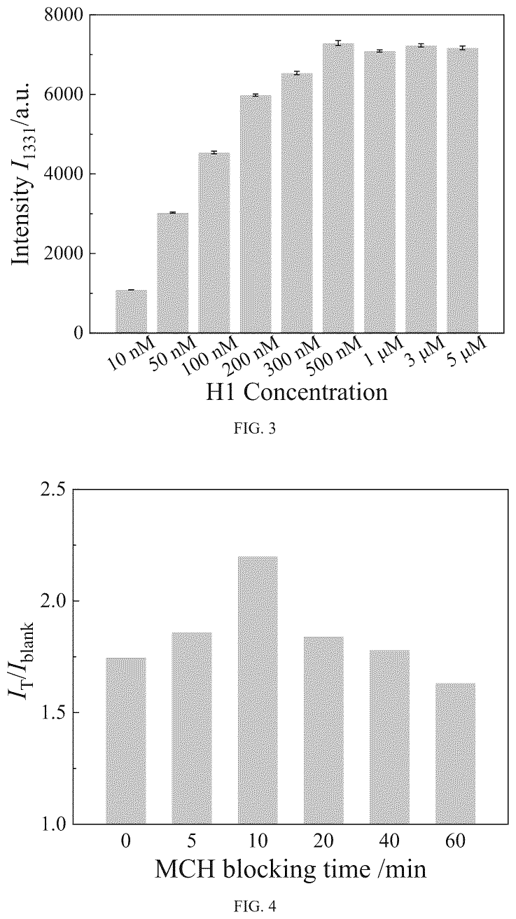

In the preparation of the SERS sensor chip, the silver nanorod array substrate is co-cultured with 20 μL of 10 nm, 50 nM, 100 nM, 200 nM, 300 nM, 500 nM, 1 μM, 3 μM, and 5 μM of hairpin DNA single-stranded H1 solution, respectively, and is placed in the constant temperature mixer at 25° C. After reaction for 3 hours, the substrate is washed with reaction buffer and DEPC water in sequence for many times to obtain the SERS sensor chip for detecting gastric cancer exosomes. Then, hairpin nucleic acid aptamer strand MUC1-apt, Cas13a protein, crRNA, uracil-modified hairpin recognition single-stranded HR, hairpin DNA single-stranded H2, and an SERS probe are mixed with 10⁶ particles·mL⁻¹ of gastric cancer exosome samples; and the mixed solution is dripped on the surface of an SERS sensor chip, and is placed in a constant temperature mixer of 300 rpm at 25° C. for 60 minutes. The reaction buffer and ultra-pure water are used to clean the pores and carry out SERS detection to obtain the SERS spectrum, as shown in FIG. 3. It can be seen that when the concentration of H1 is less than 500 nM, the SERS intensity increases monotonously. When the concentration of H1 is greater than 500 nM, SERS intensity reaches the saturation state, which indicates that the optimal incubation concentration of H1 is 500 nM.

As for optimization of 6-mercaptoethanol (MCH) blocking time on the surface of the SERS sensor chip

In the preparation of the SERS sensor chip, the silver nanorod array substrate is co-cultured with 20 μL of 500 nm of hairpin DNA single-stranded H1 for 3 hours. After the substrate is washed with reaction buffer, 20 μL of 10 μM of 6-mercaptoethanol (MCH) aqueous solution is dripped onto the surface of the substrate and placed in a constant temperature mixer at 37° C. for reaction for 0, 5, 10, 20, 40 and 60 minutes, respectively. Reaction buffer and DEPC water are used to wash the substrate for many times to obtain the SERS sensor chip, and then the SERS detects a blank sample and 10⁶ particles·mL⁻¹ of exosomes to obtain the ratio of the SERS intensity of the target exosomes to that of the blank sample (I_{T}/I_{blank}), as shown in FIG. 4. It can be seen that the maximum ratio of the SERS intensity (I_{T}/I_{blank}) is obtained when the MCH blocking time is 10 minutes, which indicated that the best blocking time of MCH aqueous solution is 10 minutes.

### Embodiment 1: The SERS Biosensor is Used to Detect Gastric Cancer Exosomes

2 μL of 10 μM of MUC1-apt, 2 μL of 0.25 μM of Cas13a protein, 2 μL of 0.25 μM of crRNA, 2 μL of 2.5 μM of HR, 2 μL of 10 μM of H2, 2 μL of gastric cancer exosomes with different concentrations (10³ to 10⁷ particles·mL⁻¹) and 5 μL of the SERS probes are taken in 20 μL of reaction buffer (10 mM of Tris-HCl, 50 mM of KCl, 1.5 M of MgCl₂, and pH 8.3), respectively.

The mixed solution of gastric cancer exosomes is not added as a blank sample. The mixed solution and the blank sample with different concentrations of gastric cancer exosomes are dripped on the surface of the SERS sensor chip, and are cultured in a constant temperature mixer of 300 rpm at 37° C. for 60 minutes. Then, the reaction buffer and DEPC water are used to clean the pores in sequence. After natural air-drying, the SERS test is carried out on the SERS sensor chip, and the SERS spectrum and the characteristic signal intensity values of gastric cancer exosomes and the blank sample with different concentrations are obtained.

Based on the complete solution of 10⁶ particles·mL⁻¹ of exosomes, incomplete mixed solution lacking a certain reagent (such as gastric cancer exosomes, crRNA or HR) is prepared for the sample as a control, and complete mixed solutions containing all reagents and complete mixed solutions of gastric cancer exosomes with different concentrations (10³ to 10⁷ particles·mL⁻¹) are prepared.

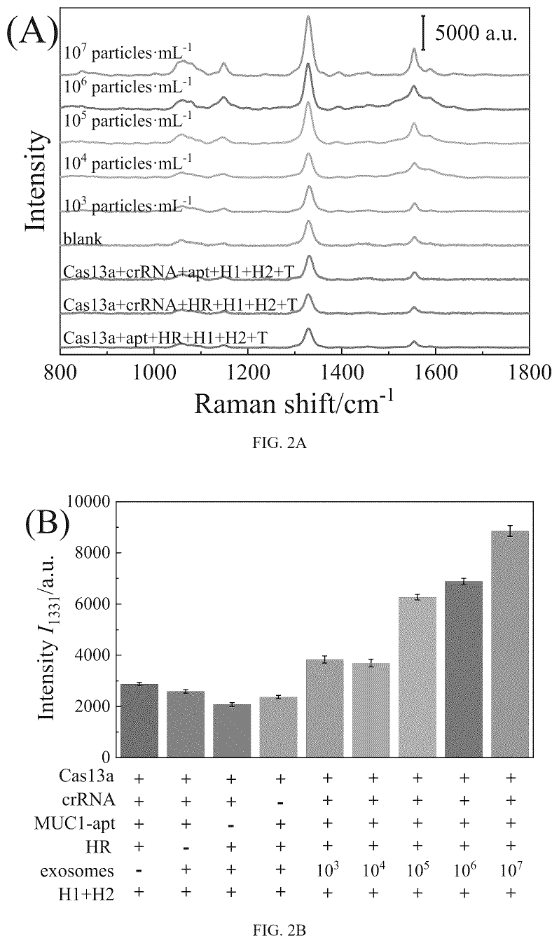

With reference to FIG. 2A and FIG. 2B, the SERS intensity measured by an incomplete detection system is the same as the signal intensity of the blank sample. In contrast, the complete mixed solution containing all reagents can effectively perform the cascade signal amplification reaction and obtain significantly enhanced SERS signal, and the SERS signal is gradually enhanced with the increase of the concentration of the target gastric cancer exosomes.

### Experimental Example 1: The Optimization Experiment of Detection Time of Detecting Gastric Cancer Exosomes by Using an SERS Biosensor

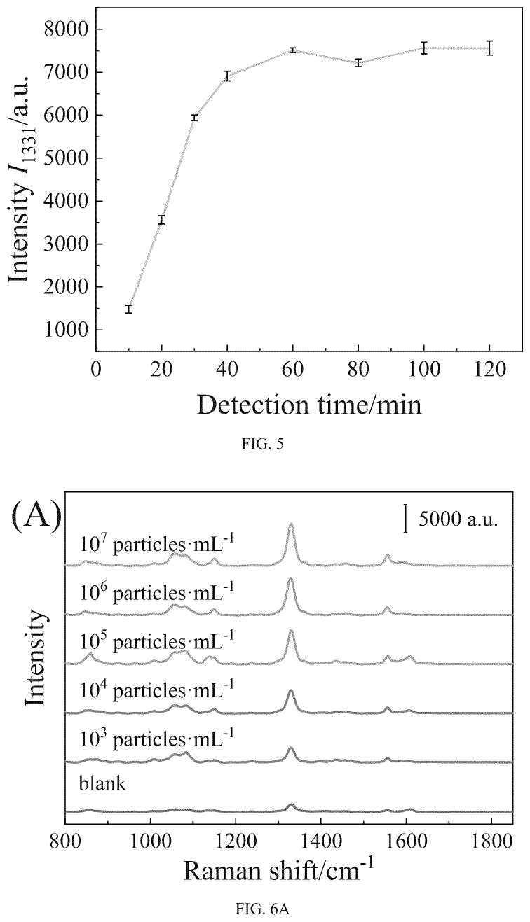

2 μL of 10 μM of MUC1-apt, 2 μL of 0.25 μM of Cas13a protein, 2 μL of 0.25 μM of crRNA, 2 μL of 2.5 M of HR, 2 μL of 10 μM of H2, 2 μL of 10⁶ particles·mL⁻¹ of gastric cancer exosomes and 5 μL of the SERS probes are added on the surface of the prepared SERS sensor chip in 20 μL of reaction buffer, and are placed in a constant temperature mixer of 300 rpm at 37° C. for 10, 20, 30, 40, 60, 80, 100 and 120 minutes. Then, the reaction buffer and DEPC water are used to clean the pores and perform SERS detection. The SERS spectrogram is obtained by the test, as shown in FIG. 5. It can be seen that by testing the SERS signal of the detection chips incubated for different time, it is observed that the SERS intensity gradually increases from 0 to 60 minutes, and the SERS signal reaches saturation state at 60 minutes of incubation, indicating that the optimal detection time of the SERS biosensor for detecting gastric cancer exosomes is 60 minutes.

### Experimental Example 2: The Working Curve and the Limit of Detection of Detecting Gastric Cancer Exosomes by Using an SERS Biosensor

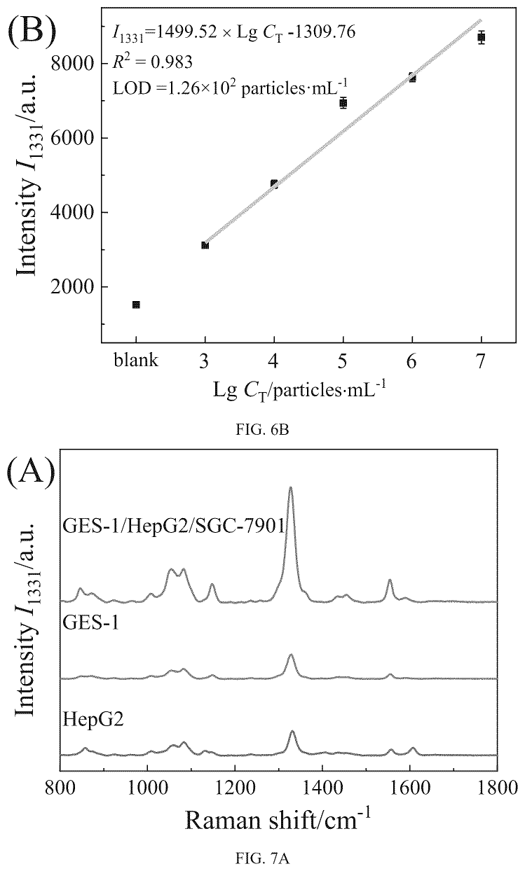

According to the optimized SERS sensor chip and the optimized detection time of Experimental Example 1, the working curve and the limit of detection of detecting gastric cancer exosomes by using an SERS biosensor are determined according to the detection process in Experimental Example 1: the gastric cancer exosomes are diluted with PBS buffer to different concentrations of 10³ to 10⁷ particles·mL⁻¹. 2 μL of 10 μM of MUC1-apt, 2 μL of 0.25 μM of Cas13a protein, 2 μL of 0.25 μM of crRNA, 2 μL of 2.5 UM of HR, 2 μL of 10 μM of H2, 2 μL of 10³ to 10⁷ particles·mL⁻¹ of gastric cancer exosomes and 5 μL of the SERS probes are added on the surface of the prepared SERS sensor chip in 20 μL of reaction buffer, and are placed in a constant temperature mixer of 300 rpm at 37° C. for 60 minutes. Then, the reaction buffer and DEPC water are used to clean the pores and perform SERS detection. The SERS spectrogram and the characteristic signal intensity value are obtained by the test. Working curves of the SERS biosensor are drawn with the logarithm of the target exosome concentration as the abscissa and the SERS characteristic peak intensity value as the ordinate, and the limit of detection of the SERS sensor of gastric cancer exosomes is calculated according to the working curves. It can be seen that FIG. 6A is the SERS spectrum obtained by detecting gastric cancer exosomes with different concentrations, and the SERS intensity increases monotonically with the increase of the target exosome concentration. FIG. 6B is an SERS peak intensity of each spectral line corresponding to the Raman shift of 1331 cm⁻¹ in FIG. 6A. The linear calibration curve is I₁₃₃₁=1499.52×LgCₑₓₒ−1309.76 (R²=0.983), and the Limit of Detection (LOD) is as low as 1.26×10² particles·mL⁻¹.

### Experimental Example 3: Performance Characterization of Detecting Gastric Cancer Exosomes by Using an SERS Biosensor

1) Specific Characterization of Detecting Exosomes from Different Cells by Using the SERS Biosensor

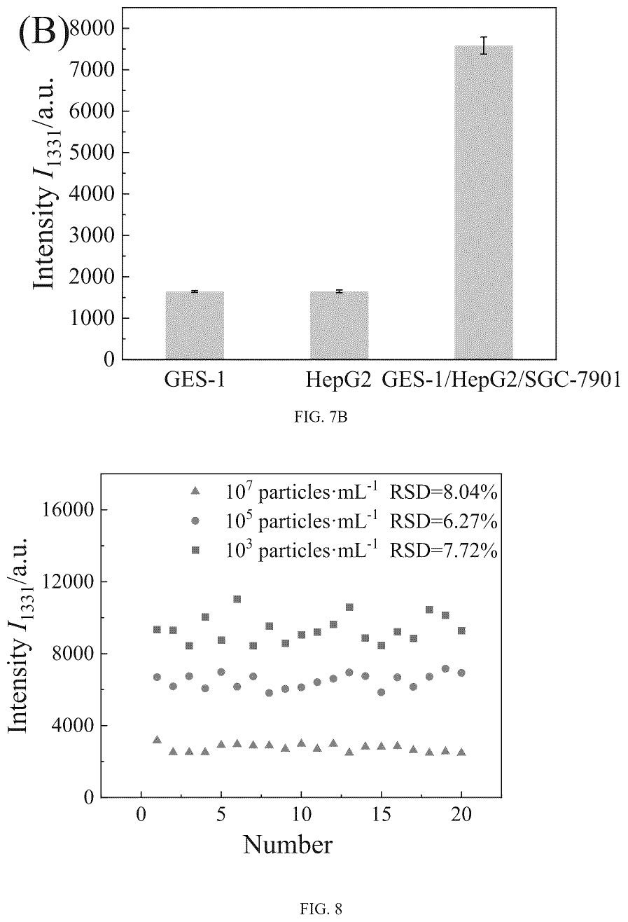

10⁶ particles·mL⁻¹ of exosomes from human normal gastric mucosa cells GES-1, exosomes from human hepatocellular carcinoma cells HepG2, and exosomes from GES-1/HepG2/SGC-7901 are prepared. 2 μL of 10 μM of MUC1-apt, 2 μL of 0.25 μM of Cas13a protein, 2 μL of 0.25 μM of crRNA, 2 μL of 2.5 μM of HR, 2 μL of 10 μM of H2, 2 μL of exosomes from different sources and 5 μL of the SERS probes are taken in 20 μL of reaction buffer, respectively, and are placed in a constant temperature mixer of 300 rpm at 37° C. for 60 minutes. Then, the reaction buffer and DEPC water are used to clean the pores and perform SERS detection. The SERS spectrogram of exosomes from different sources and the characteristic signal intensity value are obtained. It can be seen that FIG. 7A is an SERS spectrogram of an SERS biosensor used to detect mixed samples of GES-1, HepG2, and GES-1/HepG2/SGC-7901. FIG. 7B is an SERS peak intensity of each spectral line corresponding to the Raman shift of 1331 cm⁻¹ in FIG. 7A. The prepared SERS biosensor can well distinguish exosomes from gastric cancer from exosomes from other cells, which indicates that the SERS biosensor has good specificity.

### 2) Uniformity Characterization of Detecting Gastric Cancer Exosomes by Using the SERS Biosensor

2 μL of 10 μM of MUC1-apt, 2 μL of 0.25 μM of Cas13a protein, 2 μL of 0.25 μM of crRNA, 2 μL of 2.5 μM of HR, 2 μL of 10 μM of H2, 2 μL of gastric cancer exosomes with different concentrations (10³, 10⁵ and 10⁷ particles·mL⁻¹) and 5 μL of the SERS probes are added on the surface of the prepared SERS sensor chip in 20 μL of reaction buffer, and are placed in a constant temperature mixer of 300 rpm at 37° C. for 60 minutes. Then, the reaction buffer and DEPC water are used to clean the pores. The SERS signals of 20 random points on the SERS sensor chip are recorded to study the uniformity of the silver nanorod array substrate with hairpin DNA single-stranded H1 modified on the surface. FIG. 8 records SERS signals of 20 random points of 10³, 10⁵, and 10⁷ particles·mL⁻¹ of gastric cancer exosomes detected by the SERS sensor chip, showing small signal fluctuation. The relative standard deviation (RSD) of the SERS intensity is less than 8.04%, indicating that the proposed SERS biosensor has good uniformity in detecting gastric cancer exosomes.

### 3) Repeatability Characterization of Detecting Gastric Cancer Exosomes by Using the SERS Biosensor

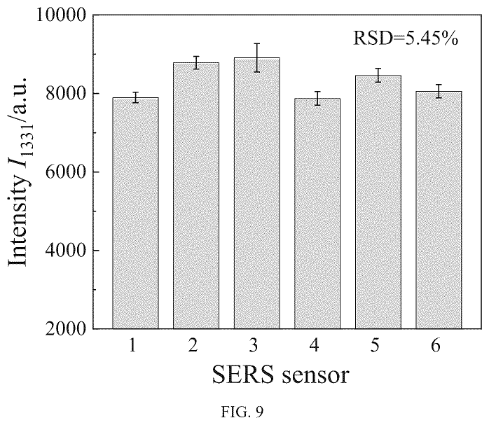

2 μL of 10 μM of MUC1-apt, 2 μL of 0.25 UM of Cas13a protein, 2 μL of 0.25 μM of crRNA, 2 μL of 2.5 UM of HR, 2 μL of 10 μM of H2, 2 μL of 10⁷ particles·mL⁻¹ of gastric cancer exosomes and 5 μL of the SERS probes are added on six sets of SERS sensor chips prepared in Embodiment 1 in 20 μL of reaction buffer, and are placed in a constant temperature mixer of 300 rpm at 37° C. for 60 minutes. Then, the reaction buffer and DEPC water are used to clean the pores. The SERS spectra of 10 random points on the different detection chips are recorded to obtain the average SERS signal intensity value. FIG. 9 records the SERS intensity of 10⁷ particles·mL⁻¹ of gastric cancer exosomes detected by six sets of different batches of the SERS sensor chips, showing a small relative standard deviation (RSD=5.45%), indicating that the proposed SERS biosensor has good repeatability in detecting gastric cancer exosomes.

What is claimed is:

1. A Surface-Enhanced Raman Scattering (SERS) biosensor based on a Clustered Regularly Interspaced Short Palindromic Repeats (CRISPR)/Cas13a system, wherein the SERS biosensor comprises an SERS sensor chip, a first reagent, a second reagent, a third reagent, and a fourth reagent;
the SERS sensor chip is a silver nanorod array substrate with hairpin Deoxyribonucleic Acid (DNA) single-stranded H1 and a blocking molecule 6-mercaptoethanol (MCH) modified on the surface;
the first reagent is an SERS probe and buffer of the probe; and the SERS Probe is a composite nanoparticle where a surface of a gold nanoparticle is modified with a Probe single strand and a Raman molecule DTNB;
the second reagent is hairpin nucleic acid aptamer strand MUC1-apt aqueous solution;
the third reagent is the CRISPR/Cas13a system and buffer of the system, and the CRISPR/Cas13a system comprises Cas13a protein, crRNA, and uracil-modified hairpin recognition single-stranded HR; and
the fourth reagent is hairpin DNA single-stranded H2 aqueous solution;
wherein the hairpin DNA single-stranded H1 base sequence is shown in SEQ ID NO. 1, the hairpin nucleic acid aptamer strand MUC1-apt base sequence is shown in SEQ ID NO. 2, the uracil-modified hairpin recognition single-stranded HR base sequence is shown in SEQ ID NO. 3, the crRNA base sequence is shown in SEQ ID NO. 4, the hairpin DNA single-stranded H2 base sequence is shown in SEQ ID NO. 5, and the Probe single-stranded base sequence is shown in SEQ ID NO. 6.

2. The SERS biosensor based on the CRISPR/Cas13a system according to claim 1, wherein the configuration concentration of the hairpin nucleic acid aptamer strand MUC1-apt aqueous solution is 5 to 20 M.

3. The SERS biosensor based on the CRISPR/Cas13a system according to claim 1, wherein the configuration concentrations of the Cas13a protein and the crRNA are both 0.1 to 2 μM, and the configuration concentration of the uracil-modified hairpin recognition single-stranded HR is 1 to 10 μM.

4. The SERS biosensor based on the CRISPR/Cas13a system according to claim 1, wherein the configuration concentration of the hairpin DNA single-stranded H2 is 5 to 20 μM.

5. The SERS biosensor based on the CRISPR/Cas13a system according to claim 1, wherein the particle size of the gold nanoparticle is 15 to 100 nm.

6. The SERS biosensor based on the CRISPR/Cas13a system according to claim 1, wherein the preparation method of the SERS sensor chip is as follows:
(1) preparing a silver nanorod array, and washing the silver nanorod array with Diethyl Pyrocarbonate (DEPC) water for many times before use;
(2) co-culturing the hairpin DNA single-stranded H1 aqueous solution with the silver nanorod array in the concentration range of 0.5 to 2 μM, and standing for 3 to 5 hours at the culturing condition of 25 to 37° C. and the humidity environment of 60 to 80%; wherein H1 is immobilized on the surface of the silver nanorod array through a covalent bond formed between a thiol group and a silver; and
(3) after cleaning the substrate with reaction buffer with pH of 8.3, dripping 10 μM of MCH aqueous solution onto a surface of the substrate, and placing MCH aqueous solution in a constant temperature mixer at 37° C. for 10 minutes; wherein a volume ratio of the hairpin DNA single-stranded H1 aqueous solution to the MCH aqueous solution is 1:1; and the components of the reaction buffer comprise 10 mM of Tris-HCl, 50 mM of KCl, and 1.5 M of MgCl₂.

7. The SERS biosensor based on the CRISPR/Cas13a system according to claim 1, wherein the silver nanorod array is prepared by a vacuum electron beam evaporation coating device and an oblique angle deposition technology, and the silver nanorod array substrate is covered with a layer of polydimethylsiloxane (PDMS) film with 3×10 pores, in which a single pore has a diameter of 4 mm and a height of 1 mm.

8. The SERS biosensor based on the CRISPR/Cas13a system according to claim 1, wherein the preparation step of the first reagent comprises:
1) mixing the Probe single strand with a Tricarboxyethyl Phosphine Solution (TCEP) according to a molar ratio of 1:1000, and placing the mixture in a constant temperature mixer at 25° C. for reaction for 4 hours;
2) mixing 50 μM of Probe single-stranded aqueous solution with 2.3 nM of AuNPs aqueous solution in Phosphate-Buffered Saline (PBS) buffer with pH 7.4, in which the components of the buffer comprise 10 mM of Na₂HPO₄, 1.76 mM of KH₂PO₄, 137 mM of NaCl, and 2.7 mM of KCl, and the mixture is placed in the constant temperature mixer at 25° C. for shaking incubation for 5 hours; and a volume ratio of the Probe single-stranded aqueous solution to the AuNPs aqueous solution is 1:50;
3) adding 10, 20, 30 and 40 μL of 2M of NaCl solution into the mixture obtained in Step 2) in sequence every 0.5 hour for shaking incubation for 5 hours; and
4) adding 100 μM of DTNB to the mixture obtained in Step 3), placing the mixture in the constant temperature mixer at 25° C. for shaking incubation for 3 hours, centrifuging at 12000 rpm for 20 minutes, and washing the mixture with the PBS buffer for three times; and finally, re-dispersing the SERS probe in the PBS buffer and storing the SERS probe at 4° C. for subsequent use.

9. An application of the Surface-Enhanced Raman Scattering (SERS) biosensor according to any one of claim 1 in preparing a detection kit for gastric cancer cell exosomes, wherein the application step comprises:
1) adding SGC-7901 cell-derived exosomes into Phosphate-Buffered Saline (PBS) buffer to prepare sample solution; wherein the concentration of exosomes in the obtained sample solution ranges from 1.44×10³ to 1.44×10⁷ particles·mL⁻¹;
2) mixing hairpin nucleic acid aptamer strand MUC1-apt, Cas13a protein, crRNA, uracil-modified hairpin recognition single-stranded HR, hairpin DNA single-stranded H2, and an SERS probe with different concentrations of gastric cancer exosome samples, and dripping the mixture on the surface of an SERS sensor chip for co-culturing; and standing for 60 minutes at the culturing condition of 25 to 37° C. and the humidity environment of 60 to 80%; and
3) washing the sample processed in Step 2) with Diethyl Pyrocarbonate (DEPC) water for many times, and then carrying out the SERS test to obtain SERS spectra of target exosomes with different concentrations, drawing working curves of SERS sensors with the logarithm of the target exosome concentration as the abscissa and the SERS intensity of the DTNB characteristic peak as the ordinate, respectively, and calculating the limit of detection of the SERS sensor of the sensor according to the working curves; and
performing culturing in a constant temperature mixer of 300 rpm in Step 2) for 60 minutes at the culturing condition of 25 to 37° C. and the humidity environment of 60 to 80%.
